(19) United States
(12) Patent Application Publication
(10) Pub. No.: US 2018/0081001 A1
(43) Pub. Date: Mar. 22, 2018

(54) Magnetic Sensor, Magnetic Sensor Device, and Diagnostic Device

(71) Applicant: Kabushiki Kaisha Toshiba, Tokyo (JP)

(72) Inventors: Hitoshi IWASAKI, Tokyo (JP); Akira KIKITSU, Yokohama (JP); Satoshi SHIROTORI, Yokohama (JP)

(73) Assignee: Kabushiki Kaisha Toshiba, Tokyo (JP)

(21) Appl. No.: 15/444,820

(22) Filed: Feb. 28, 2017

(30) Foreign Application Priority Data
Sep. 20, 2016 (JP) 2016-182935

(51) Int. Cl. G01R 33/09; G01R 33/00; A61B 5/04; A61B 5/055; A61B 5/00

(52) U.S. Cl. CPC G01R 33/093; G01R 33/0029; A61B 5/04007; A61B 5/055; A61B 5/6803; A61B 5/04008

(57) ABSTRACT

In one embodiment, a first magnetoresistive effect element, a current supply unit and a detecting unit is provided. The first magnetoresistive effect element is provided between first and second electrodes and along a first direction which is a current flowing direction between the first and the second electrode. The first magnetoresistive effect element includes first and second magnetic layers and a first intermediate layer provided between the first and the second magnetic layer and along the first direction and a second direction orthogonal to the first direction. The current supply unit is connected to the first and the second electrode and can supply an alternating current. The detecting unit detects a second harmonic component of an alternating current voltage signal outputted from the first magnetoresistive effect element. A length of the first magnetoresistive effect element in the first direction is larger than a length in the second direction.

### CROSS-REFERENCE TO RELATED APPLICATION

This application is based upon and claims the benefit of priority from the prior Japanese Patent Application No. 2016-182935, filed on Sep. 20, 2016, the entire contents of which are incorporated herein by reference.

### FIELD

Embodiments described herein relate generally to a magnetic sensor, a magnetic sensor device and a diagnostic device.

### BACKGROUND

A magnetic sensor to which magnetoresistive effect elements are applied is proposed. The magnetic sensor is desired to have higher detection sensitivity.

### BRIEF DESCRIPTION OF THE DRAWINGS

FIGS. 1A and 1B are a top view and a sectional view of a main body of a magnetic sensor according to a first embodiment.

FIG. 2 is a view illustrating a configuration of a magnetoresistive effect element of the main body of the magnetic sensor, and a relationship between a current direction of the magnetoresistive effect element and a magnetic field direction of a free layer.

FIG. 3 is a view illustrating a relationship between a current magnetic field H and a resistance R in the magnetic sensor.

FIGS. 4A and 4B are views respectively illustrating a relationship between a cycle of an alternating current and a voltage corresponding to the resistance R in the magnetic sensor according to the first embodiment.

FIGS. 5A and 5B are views respectively illustrating a second harmonic signal produced in proportion to positive and negative signal magnetic fields of the magnetic sensor.

FIGS. 6A and 6B are circuit block diagrams of detecting units which detect the second harmonic signal in the magnetic sensor, respectively.

FIG. 7A is a top view of a main body of a magnetic sensor according to a second embodiment.

FIG. 7B is a view illustrating a circuit example including the main body of the magnetic sensor according to the second embodiment.

FIG. 8A is a view illustrating an arrangement seen from a vertical direction with respect to a substrate surface of a magnetic sensor according to a third embodiment.

FIGS. 8B and 8C are views illustrating arrangement examples of sensor groups composing the magnetic sensor illustrated in FIG. 8A, respectively.

FIG. 8D is a view illustrating a magnetic sensor according to a modified example of the third embodiment.

FIG. 9A is a top view illustrating a configuration of a main body of a magnetic sensor according to a fourth embodiment.

FIGS. 9B and 9C are sectional views of the main body of the magnetic sensor shown in FIG. 9A, respectively.

FIG. 9D is a view illustrating magnetoresistive effect elements connected in series in the magnetic sensor.

FIG. 10A is a top view illustrating a configuration of a main body of a magnetic sensor according to a fifth embodiment.

FIGS. 10B and 10C are sectional views of the main body of the magnetic sensor in FIG. 10A, respectively.

FIG. 11 is a view illustrating a configuration of the main body of the magnetic sensor according to the sixth embodiment, and a relationship between a current direction and a magnetic field direction of a free layer of a magnetoresistive effect element.

FIG. 12 is a view illustrating a relationship between a current magnetic field and a resistance in the magnetic sensor according to the fifth embodiment.

FIGS. 13A to 13C are views illustrating temporal changes in resistances in the magnetic sensor according to the fifth embodiment, respectively.

FIG. 14 is a view illustrating a configuration example where the magnetic sensor is applied to a magnetoencephalography and a diagnostic device.

FIG. 15 is a view illustrating another example where the magnetic sensor is applied to the magnetoencephalography.

FIG. 16 is a view illustrating an example where the magnetic sensor is applied to an electrocardiograph.

### DETAILED DESCRIPTION

According to one embodiment, a magnetic sensor having a first electrode, a second electrode, a first magnetoresistive effect element, a current supply unit and a detecting unit is provided. The first magnetoresistive effect element is provided between the first electrode and the second electrode and along a first direction which is a current flowing direction between the first electrode and the second electrode. The first magnetoresistive effect element includes a first magnetic layer, a second magnetic layer and a first intermediate layer which is provided between the first magnetic layer and the second magnetic layer and along the first direction and a second direction orthogonal to the first direction. The current supply unit is connected to the first electrode and the second electrode and can supply an alternating current. The detecting unit detects a second harmonic component of an alternating current voltage signal outputted from the first magnetoresistive effect element. A length of the first magnetoresistive effect element in the first direction is larger than a length in the second direction.

Hereinafter, a plurality of further embodiments will be described with reference to the drawings. In the drawings, the same reference numerals denote the same or similar portions respectively.

The drawings are schematic or conceptual, and a relation between the thickness and the width of each portion, and a size ratio of portions are not necessarily the same as an actual relation and size ratio. Even for the same portions, a different dimension and ratio may be illustrated depending on the drawings. In graphs, normalized values are shown in a case that any unit of horizontal or vertical axis is not mentioned.

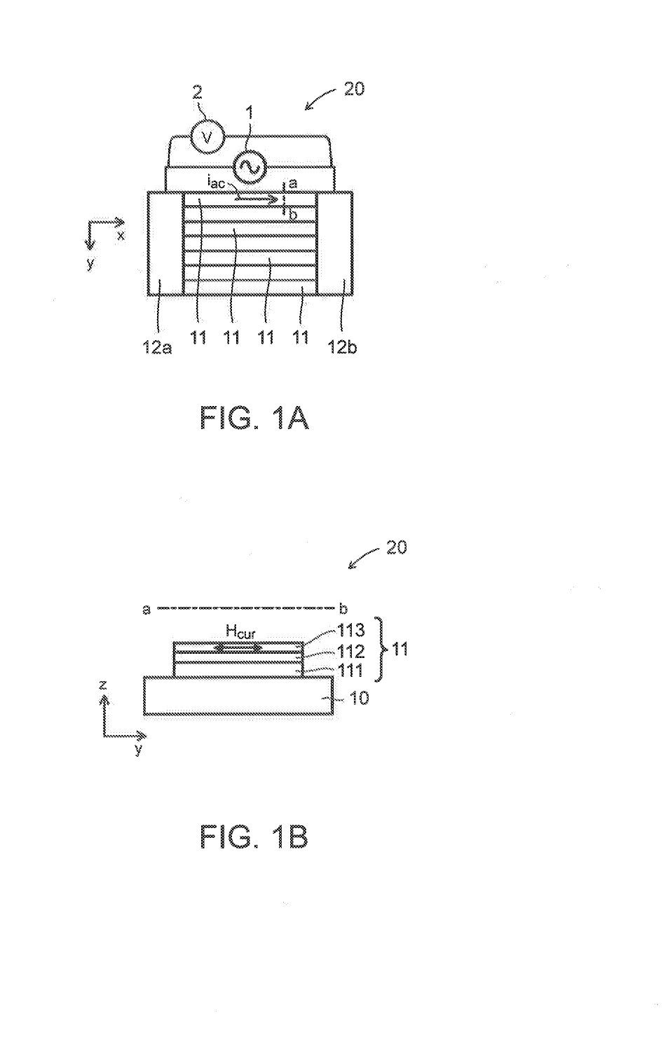

A magnetic sensor according to a first embodiment will be described with reference to FIGS. 1A and 1B.

FIG. 1A is a top view of a main body of the magnetic sensor 20. More specifically, FIG. 1A is a view illustrating the main body of a magnetic sensor 20 which is arranged on a substrate 10 of FIG. 1B and seen from an upper side of the substrate 10. FIG. 1B is a sectional view illustrating representative one of magnetoresistive effect elements 11 of FIG. 1A and taken along an a-b shown in FIG. 1A.

As illustrated in FIG. 1A, a plurality of magnetoresistive effect elements 11 patterned in a stripe shape, i.e., a rectangular shape is arranged in parallel and adjacently between an electrode 12a and an electrode 12b.

In FIG. 1A, an x-axis direction is a first direction which is a longitudinal direction of the magnetoresistive effect elements 11.

A y-axis direction is a second direction which is a width direction of the magnetoresistive effect elements 11. A z-axis direction is a third direction which is a direction vertical to film surfaces of the magnetoresistive effect elements 11, i.e., a thickness direction. In the embodiment, each magnetoresistive effect element 11 has a smaller length W in the y-axis direction (the width direction) than a length L in the x-axis direction (the longitudinal direction).

Further, each magnetoresistive effect element 11 is formed as a thin film, and accordingly has the longer length L in the x-axis direction than the thickness which is a length T in the z-axis direction. An alternating current power supply 1 and a voltmeter 2 are connected between the electrode 12a and the electrode 12b. The alternating current power supply 1 is a current supply unit. The voltmeter 2 detects resistances values of the magnetoresistive effect elements 11.

In FIG. 1A, the electrode 12a is joined to one ends of the magnetoresistive effect elements 11 in a left direction along the longitudinal direction. The electrode 12b is joined to the other ends of the magnetoresistive effect elements 11 in a right direction along the longitudinal direction. The alternating current power supply 1 causes an alternating current iac to flow in the longitudinal direction (the x-axis direction) of the magnetoresistive effect elements 11 via the electrodes 12a, 12b.

Each magnetoresistive effect element 11 has at least three layers composed of a magnetic layer 111, a non-magnetic intermediate layer 112 and a magnetic layer 113. The magnetic layer 111 is provided on the substrate 10. The non-magnetic intermediate layer 112 is provided on the magnetic layer 111. The magnetic layer 113 is provided on the non-magnetic intermediate layer 112. The magnetic layer 111 is a pinned layer whose magnetization is fixed to have the longitudinal direction (the x-axis direction), and the magnetic layer 113 is a free layer whose magnetization is rotated by a signal magnetic field H_{sig} from an outside of the magnetic sensor 20. Each magnetoresistive effect element 11 has the length L in the longitudinal direction sufficiently larger than the length W in the width direction. When the length L in the longitudinal direction is ten times the length W in the width direction or more, the free layer 113 magnetizes stably in the longitudinal direction without H_{sig}. In the embodiment, a plurality of the magnetoresistive effect elements 11 which has the stripe shape seen from above are used for detecting magnetic field. Consequently, a volume of the plurality of the magnetoresistive effect elements 11 as a whole for detecting becomes large as a whole, 1/f noise and magnetic noise due to thermal fluctuation are reduced desirably. However, a single magnetoresistive effect element 11 may be used.

Magnetization of the magnetic layer 111 is sufficiently fixed by disposing an antiferromagnetic film such as an IrMn film on the opposite surface to the surface between the magnetic layer 111 and the non-magnetic intermediate layer 112, or by sandwiching a layer such as a Ru layer between layers composing the magnetic layer 111 so that the layers are laminated. The layer such as a Ru layer causes antiferromagnetic inter-layer coupling. It is desirable to use a CoFe alloy which is suitable to exhibit a magnetoresistive effect of the magnetic layer 111. It is desirable to provide an underlayer such as Ta, Ru or a NiFeCr alloy at a side of the antiferromagnetic film on a side of the substrate 10 to improve crystalline properties, i.e., to increase diameters of crystalline particles and crystalline orientation in a direction vertical to the film surface. A material such as a CoFe alloy, a NiFe ally, a CoFeNi alloy or a laminated structure of CoFe and NiFe may be used for the magnetic layer 113. A material such as copper (Cu) which is suitable to exhibit a magnetoresistive effect may be used for the intermediate layer 112.

A magnetic field H_{cur} produced by the alternating current i_{ac} flowing in each magnetoresistive effect element 11 is applied to the width direction (the y-axis direction), and becomes a large value at the free layer 113 which exists at an upper end of each magnetoresistive effect element 11. In a case that the width of each magnetoresistive effect element 11 is approximately 1 μm, it is possible to apply a current magnetic field of approximately 50 Oe to the free layer 113 by supplying an alternating current of 5 mA from the alternating current power supply 1, which corresponds to producing a current density of approximately 50 MA/cm₂.

The current magnetic field H_{cur} applied in the width direction (the y-axis direction) plays a role of rotating the magnetization of the magnetic layer 113 in the width direction (the y-axis direction). An element width is desirably 0.5 to 5 □m to apply an effective current magnetic field to the free layer. A desirable thickness of each magnetoresistive effect element 11 in the z-axis direction is about 8.9 to 14.9 nm. Specifically, the thickness of the magnetic layer (the free layer) 113 can be 2 to 5 nm, the thickness of the intermediate layer 112 can be 2 to 3 nm, and the thickness of the magnetic layer (the pin layer) 111 can be 4.9 to 6.9 nm. In this case, the magnetic layer 111 can be laminated layers of a CoFe layer of a thickness of 2 to 3 nm, a Ru layer of a thickness of 0.9 nm or below and a CoFe layer of a thickness of 2 to 3 nm. In the embodiment, when a current direction switches, a current magnetic field Ho is applied in an opposite direction.

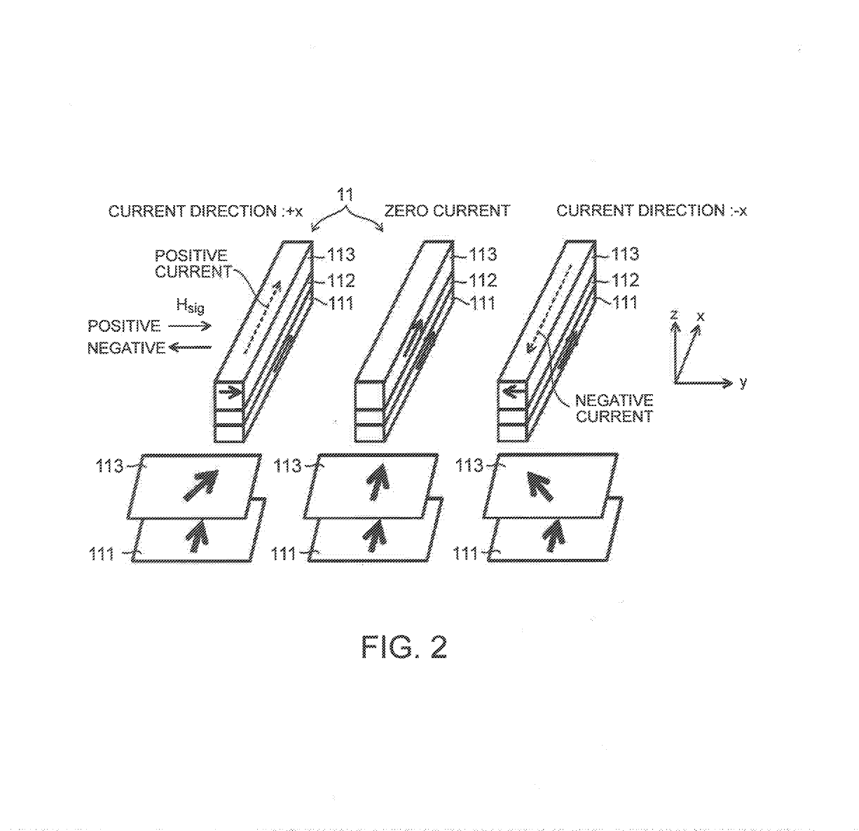

FIG. 2 is a view illustrating a configuration of a magnetoresistive effect element which is used for the magnetic sensor 20 illustrated in FIGS. 1A and 1B, and a relationship between the current direction and a magnetic field direction of the free layer in the magnetoresistive effect element.

A left side portion of FIG. 2 illustrates a case that an alternating current flows in a positive current direction (+x direction). A center portion of FIG. 2 illustrates a case that the alternating current is zero. A right side portion of FIG. 2 illustrates a case that the alternating current flows in a negative current direction (−x direction).

In the case of the left and right side portions of FIG. 2, magnetic field directions of currents applied to the magnetic layer 111 and the magnetic layer 113 are opposite to each other, and, when the current magnetic fields are increased, the magnetization of the magnetic layer 113 rotates in the width direction (the y-axis direction). The left and right side portions of FIG. 2 illustrate an example where the magnetization rotates approximately ±45 degrees. A weak current which causes slight heat generation is used to set a current value such that a rotation amount of the magnetization caused by the current magnetic field of the magnetic layer 113 is within a range of linear response.

In the case of the center portion in FIG. 2, the alternating current is zero and the magnetization of the magnetic layer 113 faces toward the same direction as the direction of the magnetization of the magnetic layer 111, which is in a low resistance state. It is possible to stabilize the magnetization of the magnetic layer 111 and the magnetic layer 113 in the same direction, by setting the thickness of the intermediate layer 112 such that positive magnetic coupling slightly occurs between the magnetic layer 111 and the magnetic layer 113.

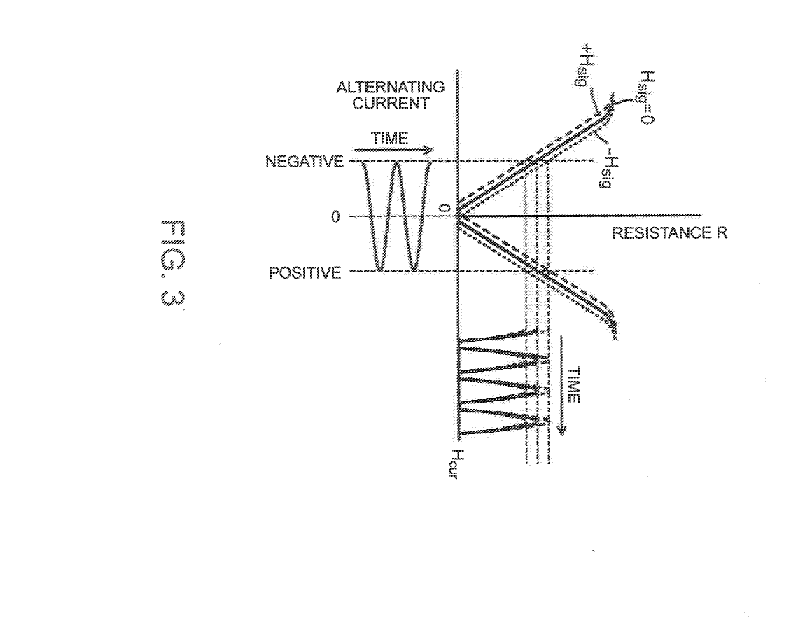

FIG. 3 is a view illustrating a relationship between a current magnetic field H which is produced by an alternating current and a resistance R of each magnetoresistive effect element 11 in the magnetic sensor 20.

More specifically, FIG. 3 illustrates a relationship between the current magnetic field H and the resistance R under presence of a positive signal magnetic field +H_{sig} from an outside of the magnetic sensor 20, a zero signal magnetic field, i.e., H_{sig}=0 and a negative signal magnetic field −Hsig from the outside. The magnetic sensor 20 uses a change in a resistance caused by a magnetic field component of each magnetoresistive effect element 11 in the width direction (the y-axis direction). Accordingly, each signal magnetic field from the outside is applied to each magnetoresistive effect element in the width direction (the y-axis direction) similar to the current magnetic field. Further, FIG. 3 illustrates a relationship between an alternating current cycle and a resistance fluctuation cycle too. Resistance increasing characteristics are symmetrical with respect to positive and negative currents under presence of the zero signal magnetic field, i.e., Hsig =0, and respective magnetization rotation angles match when absolute values of the positive and negative currents are the same. When the absolute values of the positive and negative currents are the same, the resistance fluctuations with respect to alternating currents denote the same value. When the positive signal magnetic field +H_{sig} is applied, the symmetrical resistance characteristics with respect to the positive and negative currents shift toward a negative current side. The magnetization rotation amount is large under presence of the positive current magnetic field, and the resistance R becomes large. The resistance R becomes low under presence of the negative current magnetic field.

When the negative signal magnetic field −H_{sig} is applied to each magnetoresistive effect element 11 in the width direction (the y-axis direction), the symmetrical resistance characteristics with respect to the positive and negative currents shift toward a positive current side. The magnetization rotation amount becomes small under presence of the positive current magnetic field, and the resistance R becomes low. The resistance R becomes large under presence of the negative current magnetic field. As a result, when a signal magnetic field is applied from the outside, the resistance values with respect to the positive and negative current magnetic fields become different from each other. The difference is proportional to an intensity of the signal magnetic field in a range of linear magnetic field-resistance characteristics.

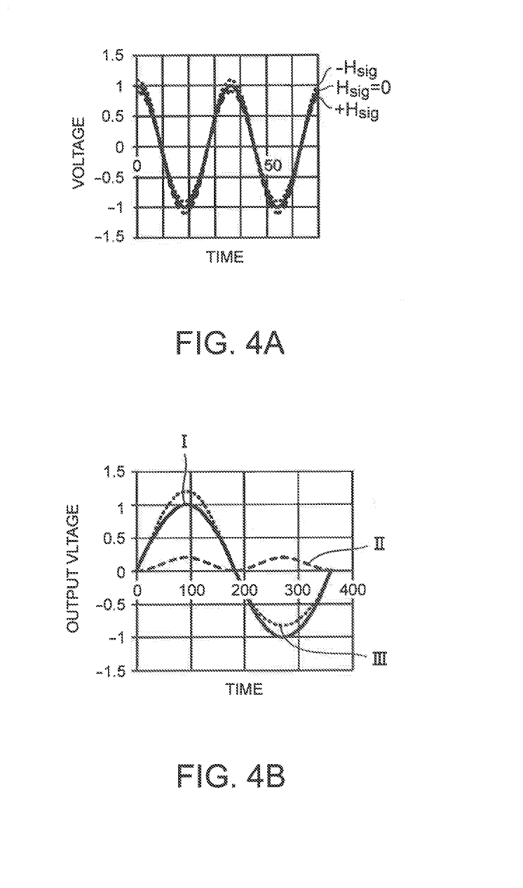

FIGS. 4A and 4B are views respectively illustrating relationships between a cycle of an alternating current and a voltage corresponding to the resistance R of each magnetoresistive effect element 11 in the magnetic sensor 20.

A voltage signal matching a current cycle is obtained under presence of the zero signal magnetic field, i.e., H_{sig}=0. When the positive signal magnetic field is applied, a voltage signal at the positive current side increases, and a signal voltage at the negative current side decreases. In contrast, when the negative signal magnetic field is applied, the voltage signal at the negative current side decreases, and the voltage signal at the positive current side increases. In FIG. 4B, a graph I shows a case in which a signal magnetic field does not exist. When the signal magnetic field is applied, a waveform formed by combining a second harmonic signal having a frequency 2f which is twice a current frequency f is produced as shown by a graph II, and a waveform formed by combining the second harmonic signal and the signal of the current frequency f is produced as shown by a graph III. The phases of the positive and negative currents differ from each other by 180 degrees. Accordingly, it is possible to detect positive and negative signal magnetic fields by detecting a second harmonic signal produced in proportion to the positive and negative signal magnetic fields together with detecting the phase, if necessary. Alternatively, it is possible to detect the positive and negative signal magnetic fields by applying a bias magnetic field which is produced by a direct current in the same direction as the direction of the signal magnetic field without detecting the phase.

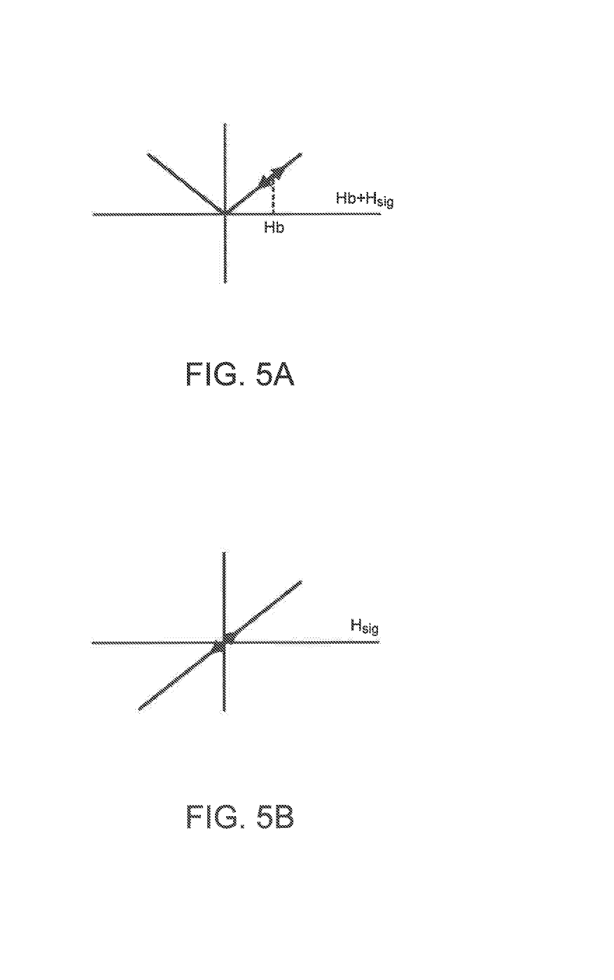

FIGS. 5A and 5B are views illustrating amplitude of a second harmonic signal produced in proportion to positive and negative signal magnetic fields of the magnetic sensor 20, respectively. The vertical axis shows the amplitude of the second harmonic signal, and the horizontal axis shows intensity of the signal magnetic fields.

As illustrated in FIG. 5A, in a case where there is a positive bias magnetic field sufficiently larger than a signal magnetic field, the second harmonic signal increases when the positive signal magnetic field is applied on the basis of a second harmonic signal produced by zero signal magnetic field. In the case, the second harmonic signal decreases when the negative signal magnetic field is applied. It is possible to apply a bias magnetic field H_{b} by superimposing a direct current of a minute amount on the alternating current and applying the superimposed current to each magnetoresistive effect element. The frequency of the alternating current is set to a value which is one digit or more higher than a frequency of the signal magnetic field. For Application to a magnetoencephalography or an electrocardiograph, the frequency of the alternating current is 1 kHz or more desirably. The frequency of the alternating current is several tens of kHz desirably when a nerve cell activity of approximately 1 kHz is detected.

Superimposing the direct current can also realize a zero state of the second harmonic signal under presence of the zero signal magnetic field. In this case, as illustrated in FIG. 5B, it is possible to obtain a voltage output by detecting the phase of the second harmonic signal and inverting the polarity of a negative second harmonic signal.

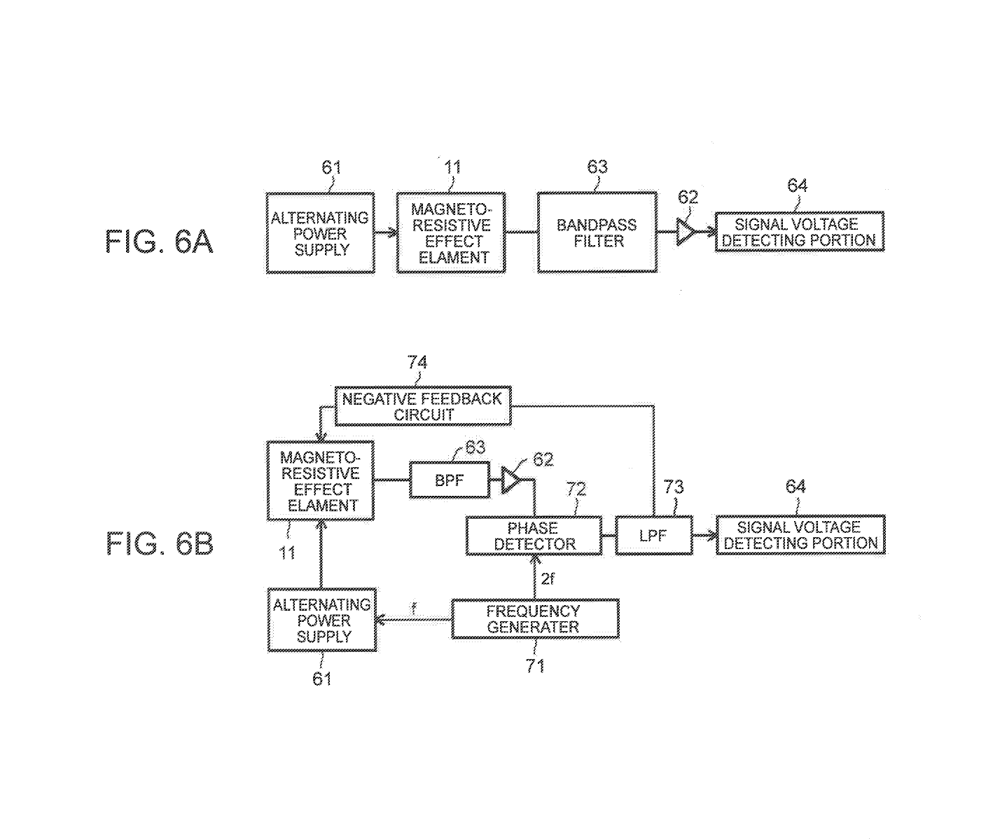

FIGS. 6A and 6B are circuit block diagrams of two detecting units which detect the second harmonic signal in the magnetic sensor 20, respectively.

FIG. 6A illustrates an example of a circuit of one of the detecting units which uses the bias magnetic field to detect a second harmonic signal and which is used when a phase is not detected. An alternating current power supply 61 generates an alternating current including a direct current offset component for applying a bias magnetic field. The alternating current power supply 61 supplies the alternating current to the magnetoresistive effect elements 11. The frequency f of the alternating current is set to a value sufficiently larger than a maximum frequency of a detected magnetic field such as a value which is one digit higher, for example. A bandpass filter 63 narrows a passband of a voltage output generated by each magnetoresistive effect element 11 to a proximity of the frequency 2f corresponding to the second harmonic signal. An amplifier 62 amplifies an amplitude voltage of the obtained second harmonic signal, and a signal voltage detecting unit 64 detects the amplitude voltage as a signal voltage. According to such a configuration, the band of the signal voltage is limited to the proximity of the frequency 2f so that an SN ratio becomes better. The sensor can operate stably by adjusting the direct current offset component and controlling the intensity of the bias magnetic field.

The detection of the second harmonic signal in the example can be regarded as detection of a difference between outputs of positive and negative current magnetic fields in the proximity of the frequency 2f. Consequently, it is possible to cancel or reduce an influence of amplitude fluctuation noise of a long-cycle such as 1/f.

FIG. 6B illustrates a circuit of the other one of the detecting units to detect a second harmonic signal. The value of the second harmonic signal which is output from the circuit is zero when an intensity of a signal magnetic field is zero. An alternating current of a frequency f is generated in an alternating current power supply 61 by using a signal of the frequency f from a frequency generator 71. The alternating current power supply 61 further adds a direct current offset component to the alternating current, and supplies the alternating current to which direct current offset component is added to each magnetoresistive effect element 11. A bandpass filter 63 has a passband in the proximity of a frequency which is twice the frequency f, and causes a voltage signal to pass through the bandpass filter 63. The voltage signal corresponds to a change in a resistance of each magnetoresistive effect element 11. Then, an amplifier 62 amplifies the voltage signal. A signal voltage detecting unit 64 detects a second harmonic signal after processing of the voltage signal in a phase detector 72 and a lowpass filter 73, which is described in detail below. It is possible to generate a second harmonic signal of substantially zero when a signal magnetic field is zero as illustrated in FIG. 5B, by adjusting the direct current offset component.

The phase detector 72 refers to a signal of the frequency 2f obtained from the frequency generator 71, and extracts a second harmonic signal produced due to distortions at a positive side and a negative side. Further, the lowpass filter 73 cancels noise of the phase detector 72. The noise cancellation enables the signal voltage detecting unit 64 to receive the second harmonic signal with an higher SN ratio. A negative feedback circuit 74 feeds back a detection signal from the lowpass filter 73 to each magnetoresistive effect element 11 so that it is possible to obtain better linear responsiveness of the second harmonic signal corresponding to a signal magnetic field. As a result, it is possible to obtain a relationship of a linear response between the signal magnetic field and the second harmonic as illustrated in FIG. 5B. The negative feedback circuit 74 may be used to adjust the direct current offset component.

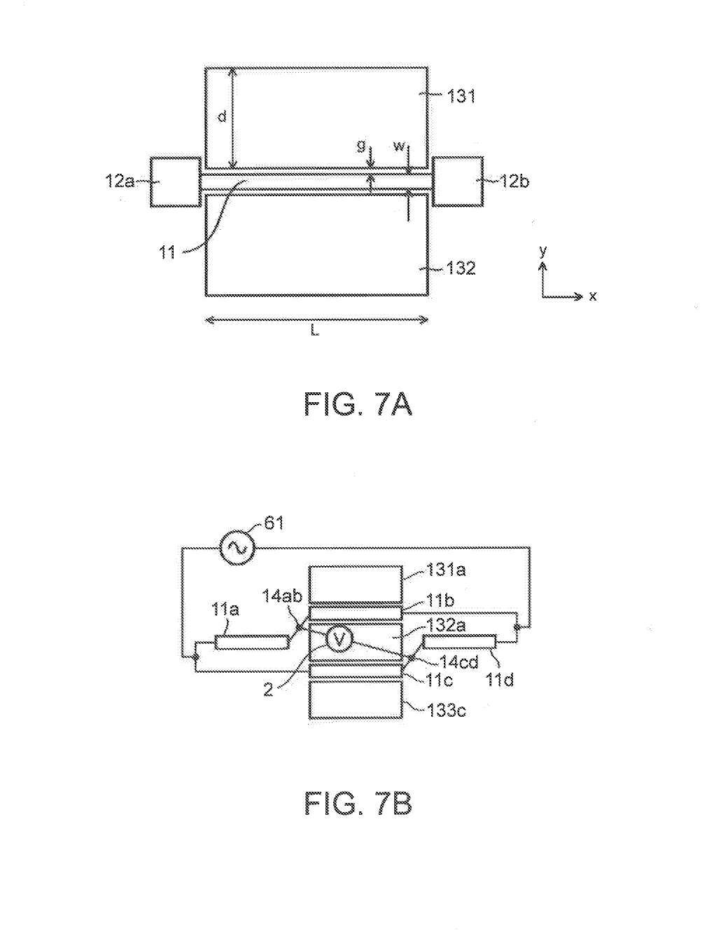

FIG. 7A is a top view of a main body of a magnetic sensor according to a second embodiment. FIG. 7B is a view illustrating a circuit example including the main body of the magnetic sensor.

In the magnetic sensor according to the second embodiment, magnetic field convergence paths 131, 132 which are close to each other with a gap g interposed between the magnetic field convergence paths 131, 132 are formed at both sides of the same magnetoresistive effect element 11 as each magnetoresistive effect element 11 of the first embodiment in a width direction (the y-axis direction). Electrodes 12a, 12b are provided at both ends of the magnetoresistive effect element 11. The magnetic field convergence paths 131, 132 are generally referred to as a magnetic flux concentrator (MFC). The magnetic field convergence paths 131, 132 provide an effect of amplifying a signal magnetic field applied to magnetic layers 111, 113 in FIG. 2B in the width direction. The magnetic field convergence paths 131, 132 are formed of a soft magnetic material such as NiFe. The soft magnetic material has a magnetization easy axis in a longitudinal direction of the magnetoresistive effect element 11 which is an x-axis direction. When d represents a width of each of the magnetic field convergence paths 131, 132, g represents a gap and w represents a width of the magnetoresistive effect element 11, an amplification factor G of a signal magnetic field can be expressed by following equation (1). The width d is approximately L/2, i.e., d˜L/2, when L is a length of the magnetic field convergence paths 131, 132 in a direction vertical to the width direction.

G˜0.6×d/(W+2g) (1)

In a case of the gap g is several nm, the width W is 0.5 to 2 μm and the width d is 0.05 to 0.5 mm, the value of the amplification factor G can be expected to be 10 to 1000. When an alternating current of 100 kHz is used and the magnetic layer 113 (the free layer) has of a length L of 100 mm and a width w of 1 μm, 1/f noise can be reduced to 10 nV/Hz close to thermal noise, for example, 0.5 nV/Hz. As a result, it is possible to detect a minute magnetic field of approximately 1 to 100 pT when 2d is 100 to 1000 mm approximately.

FIG. 7B illustrates an example in which the main body of the magnetic sensor according to the second embodiment is applied to a bridge configuration. In the example, four magnetoresistive effect elements 11a to 11d are used. A series circuit of the magnetoresistive effect elements 11a, 11b and a series circuit of the magnetoresistive effect elements 11c, 11d are connected to an alternating current power supply 1 in parallel so as to cause an alternating current to flow through the respective series circuits. Magnetic field convergence paths 131a, 132a are arranged on both sides of the magnetoresistive effect element 11b in the width direction and close to the magnetoresistive effect element 11b. The magnetic field convergence path 132a and a magnetic field convergence path 133a are arranged on both sides of the magnetoresistive effect element 11c in the width direction and close to the magnetoresistive effect element 11c. The voltmeter 2 detects a potential difference of a second harmonic signal between an intermediate point 14ab and an intermediate point 14cd. The intermediate point 14ab is provided between the magnetoresistive effect element 11a and the magnetoresistive effect element 11b. The intermediate point 14cd is provided between the magnetoresistive effect element 11c and the magnetoresistive effect element 11d.

According to such a configuration, a signal magnetic flux amplified by the magnetic field convergence paths 131 to 133a is applied only to the magnetoresistive effect elements 11b, 11c. A magnetic field which is one digit or more smaller than the amplified signal magnetic fields is applied to the magnetoresistive effect elements 11a, 11d. As a result, the potentials at the intermediate points 14ab, 14cd match with each other when the signal magnetic field is zero, and fluctuate in opposite directions when a signal magnetic field is applied. When the potential at the intermediate point 14ab is positive, the potential at the intermediate point 14cd is negative. When the potential at the intermediate point 14ab is negative, the potential at the intermediate point 14cd is positive. Accordingly, a potential difference occurs between the intermediate points 11ab and 11cd according to the signal magnetic field intensity.

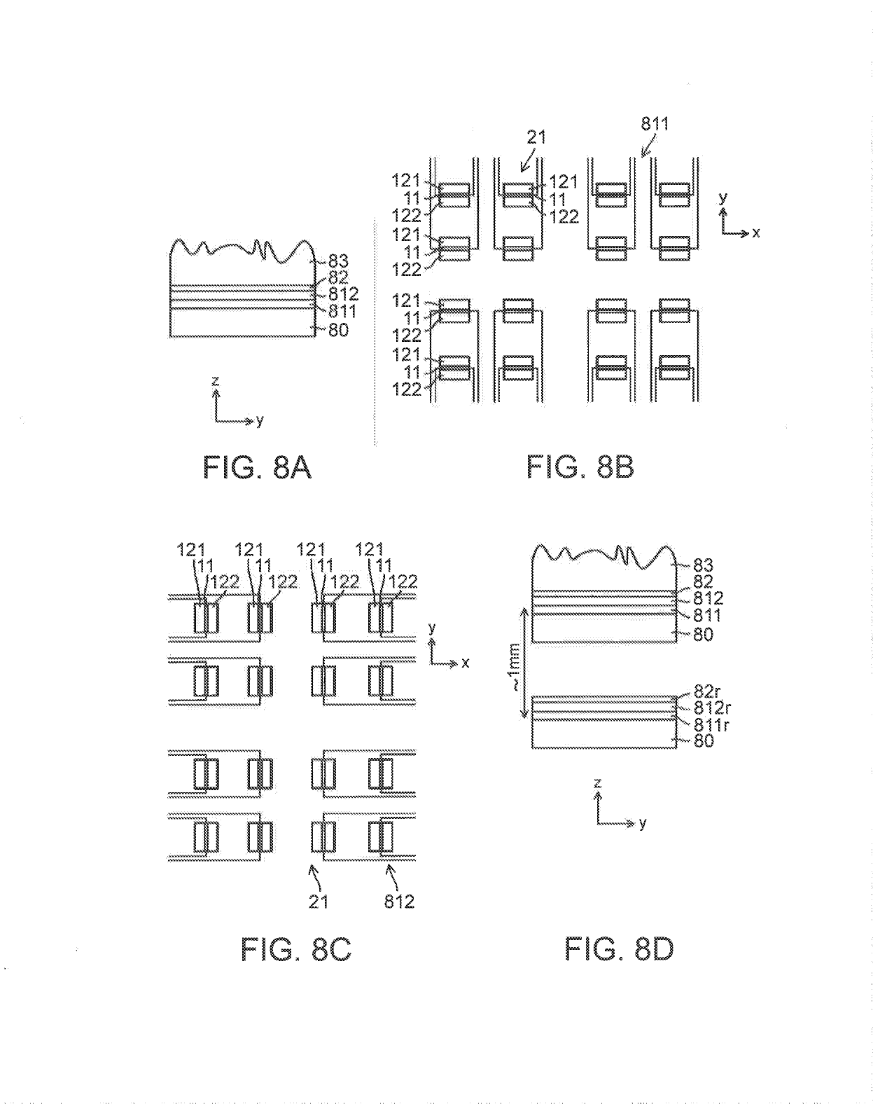

FIGS. 8A to 8C are views illustrating a configuration of a main body of a magnetic sensor according to a third embodiment which detects a magnetic field produced by an electrical activity of myocardium or nerves. FIG. 8A illustrates an arrangement when the main body of the magnetic sensor is seen from a vertical direction with respect to a substrate surface. FIG. 8B illustrates an intra-plane arrangement of a first sensor group composing the main body of the magnetic sensor. FIG. 8C illustrates an intra-plane arrangement of a second sensor group composing the main body of the magnetic sensor.

In FIG. 8A, a first sensor group 811 is formed on a substrate 80, and a second sensor group 812 is arranged on the first sensor group 811 at a narrow interval of approximately several μm. An insulation cap layer 82 such as SiOx which is suitable for cell culturing is provided on the second sensor group 812. The thickness of the insulation cap layer 82 is 1 μm or less. Further, cultured or acutely sliced myocardium or nerve cells 83 are formed on the insulation cap layer 82. The first sensor group 811 and the second sensor group 812 includes sensor units 21, for example, sixteen (16) sensor units 21 in a plane composing each of the sensor groups 811, 812.

Magnetic flux convergence paths 121, 122 which are similar to the magnetic flux convergences 131, 132 illustrated in FIG. 7A are provided on both sides of magnetoresistive effect elements 11 in each sensor unit 21. Each sensor unit 21 has an intra-plane shape of approximately 0.1 to 0.5 mm square. A longitudinal direction (the x-axis direction) of the magnetoresistive effect elements 11 of the first sensor group 811 of FIG. 8B is orthogonal to that of the magnetoresistive effect elements 11 of the second sensor group 812 of FIG. 8C. Transparent portions which allow light to pass to some degree may be provided between the sensor units 21, and may be arranged in parallel to sensors which sense fluorescence.

The first sensor group 811 detects magnetic field components in a y-axis direction in FIG. 8B and the second sensor group 812 detects magnetic field components in the x-axis direction in FIG. 8C. Consequently, it is possible to determine an intra-plane direction of a magnetic field produced from the cells 83, on the basis of an output ratio of the first sensor group 811 and the second sensor group 812. The first sensor group 811 and the second sensor group 812 can be formed in the same plane, but limitation arises to arranging the sensor units 21 densely and to enhancing the resolution. The above-described magnetic field detection provides advantages that it is possible to learn vector information such as a two-dimensional electrical signal propagation direction and an integration amount of a cell current, similarly to comparison between electrocardiograms and a magnetocardiography. A magnetic field from cells may be detected by providing the cells on a substrate different from a substrate of the magnetic sensor and placing an uppermost surface of the main body of the magnetic sensor close to the cells from an upper side of the cells.

FIG. 8D illustrates a modified example of the third embodiment. The modified example employs a configuration in which reference sensor groups 811r, 812r having a configuration similar to that of the sensor groups 811, 812 are arranged on another substrate 80 at a lower side of the main body of the magnetic sensor of FIG. 8A

A laminated body including the other substrate 80, the reference sensor groups 811r, 812r and an insulation cap 82r is provided apart from the main body of the magnetic sensor at a substantially larger interval than an interval of several mm between cells 83 and the main body of the magnetic body, for example, at an interval of approximately 1 mm. A difference between output signals of the reference sensors 811r, 812r and the sensor groups 811, 812 arranged above is detected as an output of the magnetic sensor. An external magnetic field such as a geomagnetism can be regarded as a uniform magnetic field in an area of an order of mm, and thus a difference output of the external magnetic field is substantially zero. On the other hand, the magnetic field from the cells 83 is hardly detected by the sensor which is apart by the order of mm. Accordingly, even when a signal magnetic field of the cells is detected on the basis of the difference, the sensitivity of the magnetic sensor slightly lowers. As a result, it is possible to reduce an influence of a disturbance magnetic field such as a geomagnetism and improve an SN ratio.

In the first embodiment, a plurality of magnetoresistive effect elements 11 is connected to an alternating current power supply 1 in parallel, and the alternating current power supply 1 supplies current to the magnetoresistive effect elements 11. There is a case where connecting the magnetoresistive effect elements 11 in parallel lowers a sensor resistance. Accordingly, a GMR sensor which supplies a direct current in a plane may employ a configuration in which magnetoresistive effect elements 11 are connected in series.

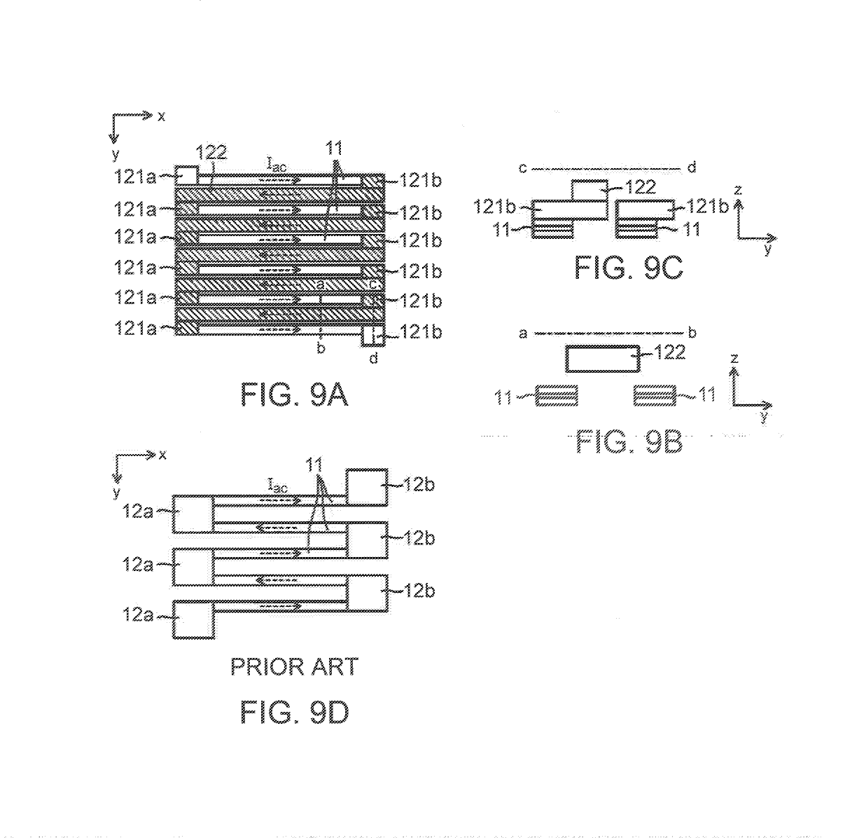

As illustrated in FIG. 9D, in a magnetic sensor in which adjacent magnetoresistive effect elements 11 are connected in series, currents of the adjacent magnetoresistive effect elements 11 flow in opposite directions. Thus, current magnetic fields applied to magnetic layers (free layers) of the adjacent magnetoresistive effect elements 11 are applied in opposite directions.

A signal magnetic field from an outside is applied in the same direction to the magnetic layers (the free layers) of the adjacent magnetoresistive effect elements 11. Consequently, an increase and a decrease in output voltages of the adjacent magnetoresistive effect elements 11 are inverted, and outputs to which these output voltages are added cancel each other. An embodiment in which magnetoresistive effect elements 11 are connected in series will be described below.

FIGS. 9A to 9C are views illustrating a configuration of a main body of a magnetic sensor according to a fourth embodiment. FIG. 9A is a top view seen from an upper side of a film surface of the main body of the magnetic sensor, i.e., from above along a z-axis direction. FIG. 9B illustrates a cross section along an a-b surface shown in FIG. 9A. FIG. 9C illustrates a cross section along a c-d surface shown in FIG. 9A.

In the fourth embodiment, each magnetoresistive effect element 11 adopts a similar structure as that of the first embodiment, but has electrodes which are different in structure from the electrodes used in the first embodiment. The main body of the magnetic sensor of the fourth embodiment has a plurality of first electrode portions 121a and a plurality of first electrode portions 121b which are arranged on a first surface including the surfaces of the magnetoresistive effect elements 11. Further, the main body of the magnetic sensor has a plurality of second electrode portions 122 arranged on a second surface including the surfaces of the first electrode portions 121a, 121b. The first electrode portions 121a, 121b are terminals which are in contact with ends of the magnetoresistive effect elements 11 in a longitudinal direction. Alternating currents are supplied to the magnetoresistive effect elements 11 from the first electrode portions 121a, 121b. The second electrode portions 122 are return current paths to align a direction in which currents flow through the magnetoresistive effect elements 11 to the same +x direction. The second electrode portions 122 are formed on the first electrode portions 121a, 121b. Such a configuration prevents a phenomenon that currents flowing in the adjacent magnetoresistive effect elements 11 in opposite directions cancel voltage outputs as described with reference to FIG. 9D. Lines of return paths of the second electrode portions 122 may be inclined from a longitudinal direction of the magnetoresistive effect elements 11. It is possible to prevent a decrease in a resistance change rate due to a magnetoresistive effect, by making the second electrode portions 122 thick using a low resistance material such as copper (Cu) so that the resistance values of the second electrode portions 122 are made sufficiently lower than those of the magnetoresistive effect elements.

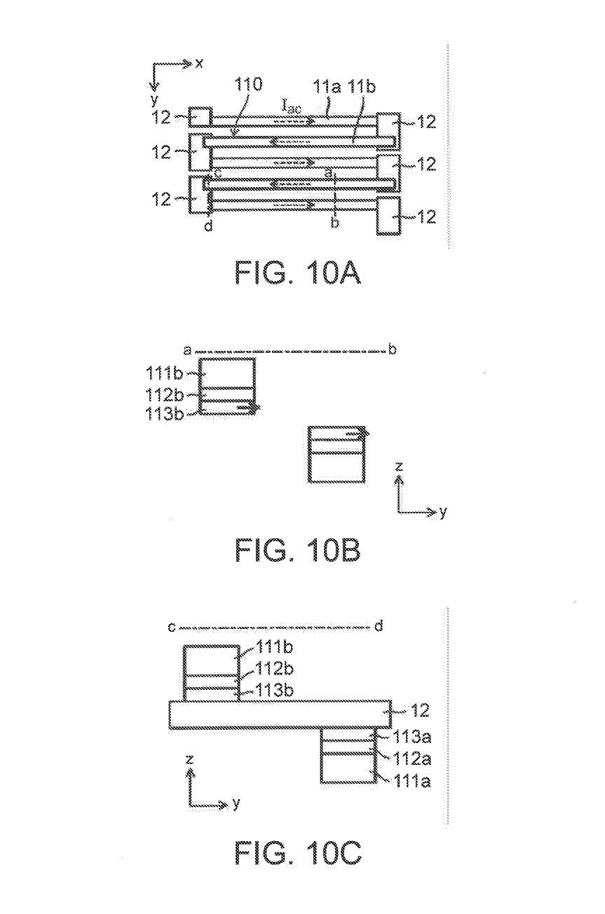

FIGS. 10A to 10C are views illustrating a configuration of a main body of a magnetic sensor according to a fifth embodiment. FIG. 10A is the top view illustrating the configuration of the main body of the magnetic sensor according to the fifth embodiment. FIG. 10B is the sectional view along an a-b surface illustrated in FIG. 10A. FIG. 10C is the sectional view along a c-d surface illustrated in FIG. 10A.

In FIG. 10A, a magnetoresistive effect element 110 of the magnetic sensor according to the fifth embodiment is composed of first element portions 11a formed on a first surface including the lower surfaces of electrodes 12 at left and right sides, and second element portions 11b formed on a second surface including the upper surfaces of the electrodes 12. Both ends of the first element portions 11a and the second element portions 11b in a longitudinal direction (the x-axis direction) are in contact with the electrodes 12 which are arranged in a middle surface between the first element portions 11a and the second element portions 11b. The first element portions 11a and the second element portions 11b are connected in series as illustrated in FIG. 10A.

An alternating current flows through the first element portions 11a in a +x direction, and an alternating current flows through a −x direction in the second element portions 11b. In FIG. 10A, a magnetic layer (a free layer) 113a of each first element portion 11a is arranged on each first electrode portion 12. An intermediate layer 112a of each first element portion 11a is arranged on the magnetic layer 113a. A magnetic layer (a pin layer) 111a of each first element portion 11a at a substrate side is provided on the intermediate layer 112a. Further, a magnetic layer (a free layer) 113b of each second element portion 11b at the substrate side is arranged on one of the electrodes 12. An intermediate layer 112b of each second element portion 11b is arranged on the magnetic layer 113b. A magnetic layer (a pin layer) 11l1b of each second element portion 11b is provided on the intermediate layer 112b. The thicknesses of the magnetic layers 111a, 111b are larger than those of the magnetic layers 113a, 113b.

Relative positions of the magnetic layers (the free layers) 113a, 113b of each first element portion 11a and each second element portion 11b are set to be opposite to each other across the one of the electrodes 12. According to such an arrangement, even when currents in the magnetic layers 113a, 113b flow in different directions, it is possible to align the current magnetic fields which are applied to the magnetic layers (free layers) 113a, 113 in the same direction, as shown by two arrows in FIG. 10B.

It is possible to prevent deterioration of characteristics of the magnetoresistive effect elements 110 such as MR ratios of the second element portions 11b, by forming the first element portions 11a and the second element portions 11b and then performing planarizing processing. Generally, it is necessary to form two kinds of magnetic films individually to change an lamination order of a pin layers and free layers of the magnetoresistive effect elements on the same surface, and a miniaturization process is difficult to be performed. However, it is easy to perform the miniaturization process by forming the first element portions 11a and the second element portions 11b of the magnetoresistive effect elements 110 on different surfaces respectively as adopted in the fifth embodiment.

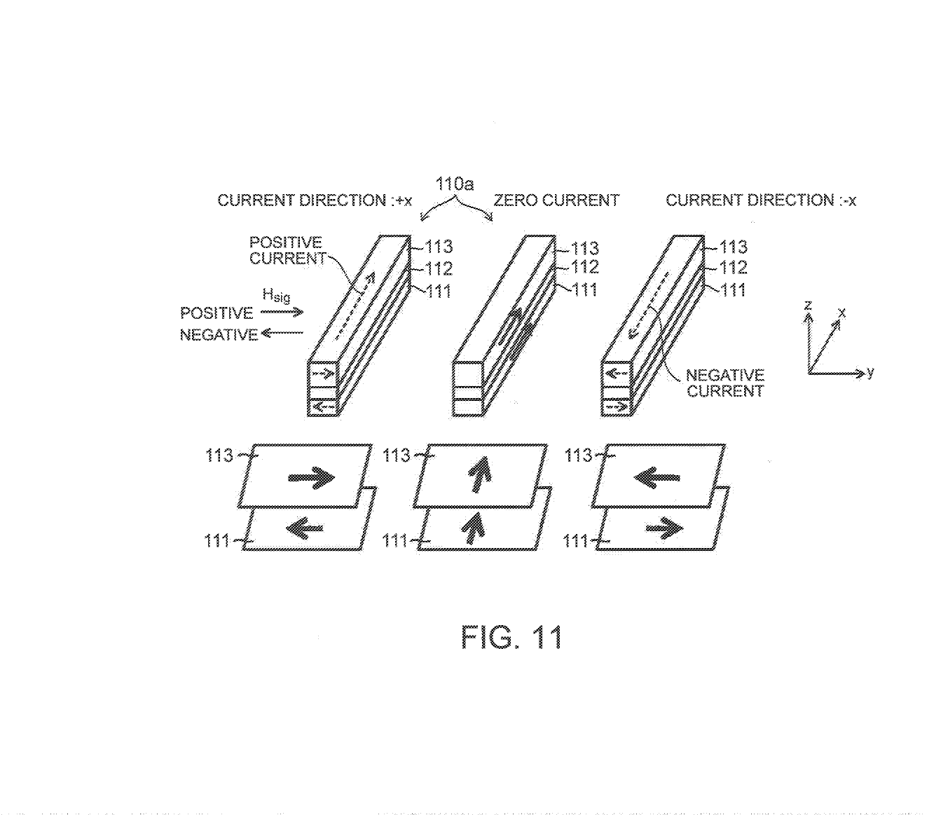

FIG. 11 is a view illustrating a configuration of the main body of the magnetic sensor according to the sixth embodiment, and a relationship between a current direction of each magnetoresistive effect element and a magnetic field direction of a pair of free layers.

The magnetic sensor according to the embodiment employs a configuration in which both of magnetic layers 111, 113 of each magnetoresistive effect element 110a are free layers whose magnetization is rotated by a current magnetic field as illustrated in FIG. 11. An intermediate layer 112 is provided between the magnetic layers 111, 113.

In the embodiment, a magnetic film thickness Mst-111 which is a product of a thickness t of the magnetic layer 111 and saturation magnetization Ms is different from a magnetic film thickness Mst-113 which is a product of the thickness t of the magnetic layer 113 and the saturation magnetization Ms. For example, a CoFe layer having a thickness of 4 nm is used for the magnetic layer 111, and a CoFe layer having a thickness of 3 nm is used for the magnetic layer 113. NiFe may be used instead of CoFe.

A current i_{ac} including a positive current and a negative current applies reverse magnetic fields to the magnetic layer 111 and the magnetic layer 113 in directions indicated by broken line arrows illustrated in FIG. 11, and the magnetization rotates in the width direction (±y direction).

In FIG. 11, the directions of current magnetic fields produced by a positive current illustrated at a left portion and a negative current illustrated at a right portion are opposite. Thus, the magnetization of the magnetic layer 111 and the magnetic layer 113 face toward the opposite directions as indicated by solid lines. The central portion illustrates magnetization when a current is zero. Copper (Cu) which provides a great magnetoresistive effect is desirably used for the intermediate layer 112. The copper has a lower resistance than resistances of the magnetic layers 111, 113 and allows a current to concentrate on the intermediate layer 112, and thus is suitable to apply large current magnetic fields to the magnetic layer 111 and the magnetic layer 113 in the opposite directions. In the embodiment, it is desirable to flow a large alternating current which saturates substantially in a width direction in a case of a maximum current magnetic field through magnetoresistive effect elements 110a of the magnetic layers 111, 113, which is different from the first embodiment.

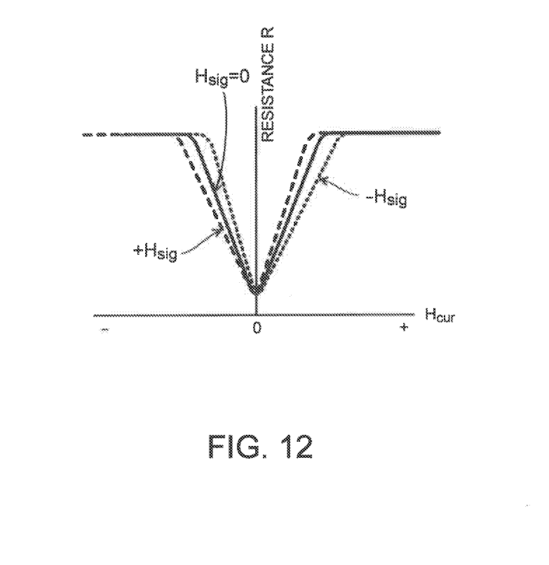

FIG. 12 is a view illustrating a relationship between a current magnetic field and a resistance in the magnetic sensor according to the fifth embodiment.

As illustrated in FIG. 12, when a signal magnetic field from an outside is zero, i.e., when H_{sig}=0 holds, resistance characteristics become more symmetrical as positive and negative current magnetic fields H_{CUR} increase more, and absolute values of the positive and negative current magnetic fields which are necessary to saturate resistances match. When a positive signal magnetic field +H_{sig} is applied from the outside, magnetization of the magnetic layer 113 whose magnetic film thickness is large easily saturates in a direction of a positive current magnetic field, and hardly saturates in a direction of a negative current magnetic field. In contrast, when a negative signal magnetic field −H_{sig} is applied from the outside, the magnetization of the magnetic layer 113 easily saturates in the direction of the negative current magnetic field. As a result, the current magnetic field which is necessary for saturation shifts in an opposite direction in a case of the positive signal magnetic field and in a case of the negative signal magnetic field. In the embodiment, a pin layer is not used, which is deferent from the first embodiment. Accordingly, a change caused by positive and negative signal magnetic fields is weak under a weak current magnetic field which does not saturate, and thus it is desirable to use a large current magnetic field which saturates. According to the embodiment, it is possible to further reduce magnetic noise by resetting a magnetic domain by using an alternating current magnetic field which saturates compared to the first embodiment.

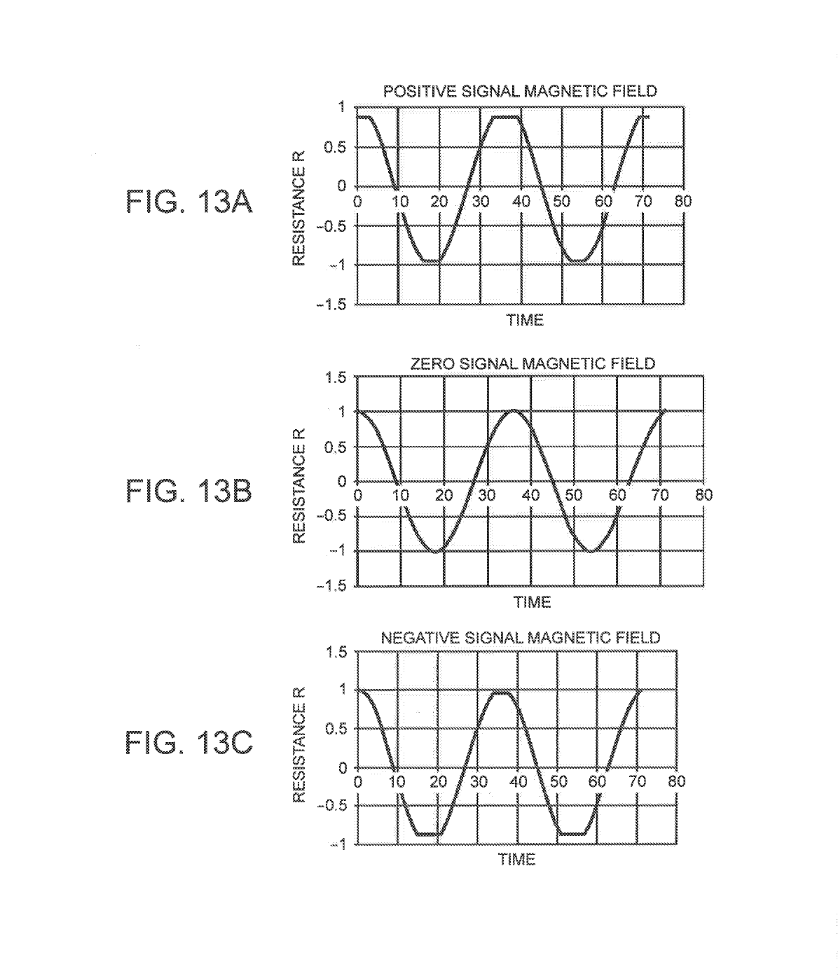

FIGS. 13A to 13C are views illustrating temporal changes in resistances in the magnetic sensor according to the sixth embodiment.

More specifically, FIGS. 13A to 13C illustrate temporal changes in resistances R under presence of a positive signal magnetic field, a zero signal magnetic field and a negative signal magnetic field.

When there is no signal magnetic field as illustrated in FIG. 13B, a resistance fluctuates according to a frequency of the alternating current i_{ac} which is supplied, and a second harmonic signal is not produced. On the other hand, when the positive signal magnetic field +H_{sig} is applied, the positive current easily distorts, and the positive current causes greater waveform distortion than that caused by the negative current. In contrast, when the negative signal magnetic field −H_{sig} is applied, the negative current causes greater waveform distortion than distortion caused by the positive current. When the positive and negative signal magnetic fields are applied, a second harmonic signal is produced according to the signal magnetic fields. The second harmonic signal can be detected by a circuit illustrated in FIG. 6A or 6B, for example. Even when the positive and negative signal magnetic fields are applied and a resistance value saturates and is fixed, an output voltage does not saturate because an alternating current is used. By using the magnetic sensor employing a bridge configuration as illustrated in FIG. 7B, it is possible to cancel a fluctuation of an output voltage under a constant resistance value and consequently to detect a second harmonic wave precisely.

The magnetic sensor according to the above-described first to fifth embodiments can be applied to a magnetoencephalography as described below. The magnetoencephalography is a device which detects a magnetic field produced by cranial nerves. When the magnetic sensor is applied to the magnetoencephalography, magnetoresistive effect elements having sizes of several mm square including magnetic flux convergence paths can be used.

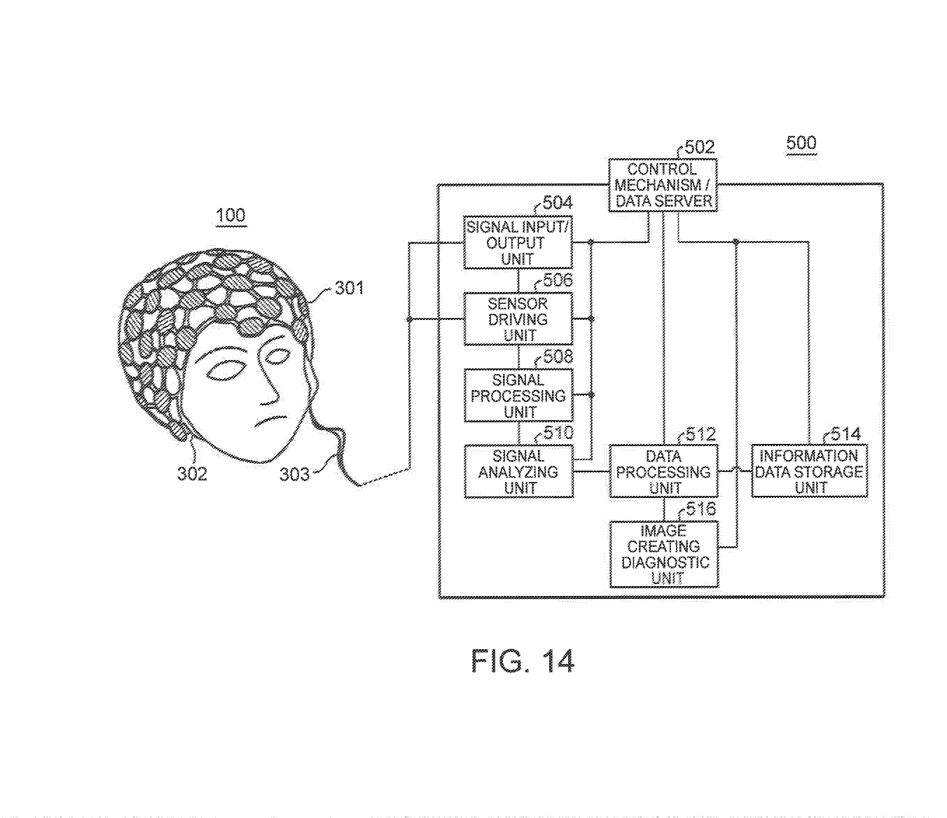

FIG. 14 is a view illustrating a configuration example where a magnetic sensor is applied to a magnetoencephalography as a magnetic sensor device and a diagnostic device. In the example, one of the magnetic sensors according to the above-described embodiments can be used.

A left side of FIG. 14 schematically illustrates a state where a magnetoencephalography 100 is attached to a head of a human body. The magnetoencephalography 100 employs a configuration where a plurality of sensor units such as 100 sensor units 301 is attached to a flexible base 302.

For example, one magnetic sensor 20 according to the first embodiment may be arranged or a plurality of magnetic sensors having the same configuration as that of the magnetic sensors 20 may be arranged in each sensor unit 301. A plurality of these magnetic sensors may configure a differential detection circuit. Other kinds of sensors such as a potential terminal and an acceleration sensor may be arranged together in each sensor unit 301. The magnetic sensor according to the first embodiment can be made very smaller than a conventional SQUID magnetic sensor, and, consequently, allow a plurality of sensor units and peripheral circuits to be arranged or coexist with other kinds of sensors. The flexible base 302 is composed of an elastic body such as a silicon resin, and is configured to be closely attached to a head by connecting sensor units 301 like a hat.

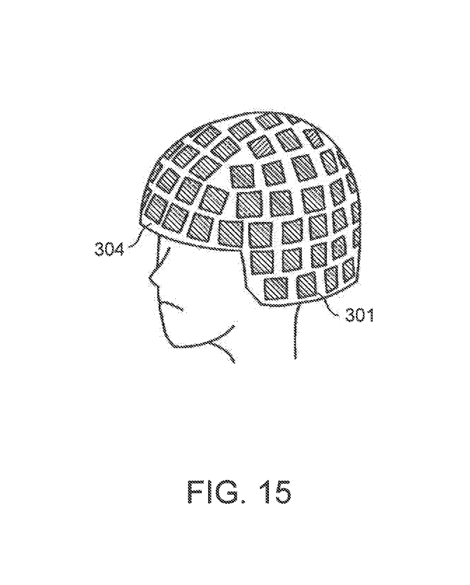

An input/output cord 303 of a plurality of sensor units 301 is connected to a sensor driving unit 506 and a signal input/output unit 504 of a diagnostic device 500. The sensor units 301 measure predetermined magnetic fields on the basis of alternating current power supplied from the sensor driving unit 506 and a control signal from the signal input/output unit 504, and the signal input/output unit 504 which is a receiving unit which receives information receives an input of a signal indicating the measurement result. The signal inputted to the signal input/output unit 504 is transmitted to a signal processing unit 508, and the signal processing unit 508 performs processing such as noise canceling, filtering, amplification and a signal arithmetic operation. The processed signal is used by a signal analyzing unit 510 to perform signal analysis for extracting a specific signal for measuring magnetoencephalo and adjusting a signal phase. Data obtained after the signal analysis is transmitted to a data processing unit 512. The data processing unit 512 performs data analysis such as neuronal firing point analysis and inverse problem analysis by receiving image data such as Magnetic Resonance Imaging (MRI) or a scalp potential information such as an electroencephalogram (EEG) from an information data storage unit 514. The data analysis result is transmitted to an image creating diagnostic unit 516, and is converted into an image which helps diagnosis. A series of operations of the signal input/output unit 504, the sensor driving unit 506, the signal processing unit 508, the signal analyzing unit 510, the data processing unit 512, the information data storage unit 514 and the image creating diagnostic unit 516 are controlled by a control mechanism/data server 502. Necessary data such as primary signal data and meta data which is under data processing are stored in the control mechanism/data server 502. As described with reference to FIG. 15 below, the data server and the control mechanism may be integrally formed.

A plurality of sensor units 301 is attached to the head of the human body in the example illustrated in FIG. 14, but may be installed at a breast of the human body. When a plurality of sensor units 301 is installed at the breast of the human body, it is possible to perform cardiac magnetic measurement. By installing a plurality of sensor units 301 at a belly of a pregnant woman, it is possible to inspect heartbeats of a fetus. It is desirable to install an overall magnetic sensor device including a subject in a shield room to prevent noise caused by geomagnetism or magnetic noise. Alternatively, a mechanism which locally shields measurement sites of the human body and a plurality of sensor units 301 may be provided. A plurality of sensor units 301 may be provided with a shield mechanism or may be effectively shielded by signal analysis or data processing.

A plurality of sensor units 301 of the magnetoencephalography 100 illustrated in FIG. 14 which includes a highly sensitive magnetic sensor is attached to the flexible base 302, butt may be attached to a fixed hard base as described below.

FIG. 15 is a view illustrating another example where a magnetic sensor is applied to a magnetoencephalography. In the example, one of the magnetic sensors according to the above-described embodiments can be used.

As illustrated in FIG. 15, a plurality of sensor units 301 is attached on a hard base 304 which has a helmet shape with a net form. The base 304 of the net form has good wearability and good adhesiveness for a human body so that it is desirable to use the base 304 of the net form. The diagnostic device 500 illustrated at the right side in FIG. 14 can be used to input signals to the sensor units 301, receive signals from the sensor units 301 and process the received signals.

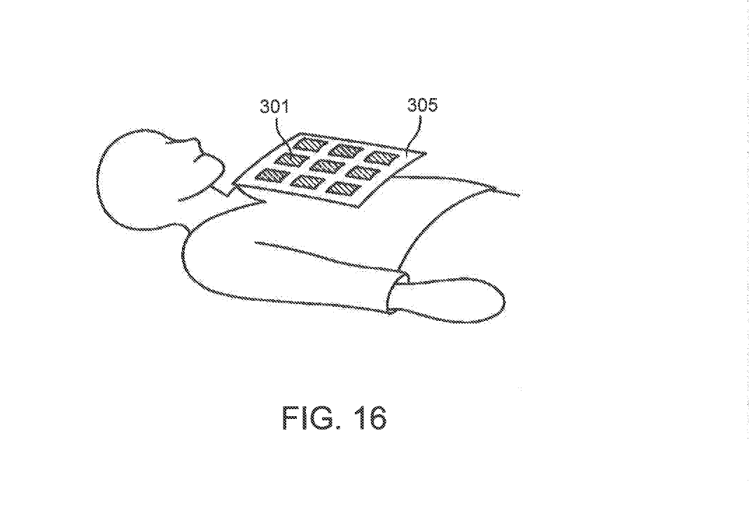

FIG. 16 is a view illustrating an example where a magnetic sensor is applied to a electrocardiograph. In the example, one of the magnetic sensors according to the above-described embodiments can be used.

As illustrated in FIG. 16, a plurality of sensor units 301 is attached on a hard base 305 of a flat shape. The diagnostic device 500 illustrated at the right side in FIG. 14 can be used to input signals to the sensor units 301, receive signals from the sensor units 301 and process the received signals.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. Forms realized by combining components of the above embodiments in a technically feasible range are included within the scope of the invention as long as the forms include the spirit of the invention.

What is claimed is:

1. A magnetic sensor comprising:
a first electrode;
a second electrode;
a first magnetoresistive effect element which is provided between the first electrode and the second electrode and along a first direction which is a current flowing direction between the first electrode and the second electrode, the first magnetoresistive effect element including a first magnetic layer, a second magnetic layer and a first intermediate layer which is provided between the first magnetic layer and the second magnetic layer and along the first direction and a second direction orthogonal to the first direction;
a current supply unit which is connected to the first electrode and the second electrode and can supply an alternating current; and
a detecting unit which detects a second harmonic component of an alternating current voltage signal outputted from the first magnetoresistive effect element,
wherein a length of the first magnetoresistive effect element in the first direction is larger than a length in the second direction.

2. The magnetic sensor according to claim 1, wherein a magnetization direction of the first magnetic layer is substantially fixed to the first direction, and a magnetization direction of the second magnetic layer is variable.

3. The magnetic sensor according to claim 1, wherein a magnetization direction of the first magnetic layer and a magnetization direction of the second magnetic layer are variable.

4. The magnetic sensor according to claim 1, wherein the detecting unit includes a bandpass filter which limits the alternating current voltage signal outputted from the first magnetoresistive effect element to a proximity of twice a frequency of the alternating current, and outputs the alternating current voltage signal to the detecting unit.

5. The magnetic sensor according to claim 1, wherein the current supply unit can further apply a direct current having a smaller current value than a current value of the alternating current.

6. The magnetic sensor according to claim 1, further comprising a fifth magnetic layer and a sixth magnetic layer,
wherein the first magnetoresistive effect element is provided along the first direction and the second direction and between the fifth magnetic layer and the sixth magnetic layer, and film thicknesses of the fifth magnetic layer and the sixth magnetic layer in a third direction orthogonal to the first direction and the second direction are larger than film thicknesses of the first magnetic layer and the second magnetic layer in the third direction.

7. The magnetic sensor according to claim 3, wherein the first and the second magnetoresistive effect elements are arranged along the second direction, and ends of the first magnetoresistive effect element and the second magnetoresistive effect element in the second direction are connected with each other, and the first magnetoresistive effect element and the second magnetoresistive effect element are connected in series.

8. The magnetic sensor according to claim 3, wherein the first magnetoresistive effect element and the second magnetoresistive effect element are provided on different surfaces along the first direction respectively, a lamination order of the first magnetic layer and the second magnetic layer and a lamination order of the third magnetic layer and the fourth magnetic layer are different, and currents which flow through the first magnetic layer and the second magnetic layer and currents which flow through the third magnetic layer and the fourth magnetic layer go in opposite directions, respectively.

9. The magnetic sensor according to claim 1, wherein the detecting unit comprising
a bandpass filter which narrows a passband of the alternating current voltage signal outputted from the magnetoresistive effect element to a proximity of twice a frequency of the alternating current,
an amplifier which amplifies an output voltage obtained from the bandpass filter, and
a signal voltage detecting unit which detects a signal voltage amplified by the amplifier.

10. The magnetic sensor according to claim 1, wherein the detecting unit comprising
a frequency generator which causes the current supply unit to generate the alternating current and outputs a signal having twice a frequency of the alternating current,
a bandpass filter which narrows a passband of the alternating current voltage signal outputted from the magnetoresistive effect element to a proximity of twice a frequency of the alternating current,
an amplifier which amplifies an output voltage obtained from the bandpass filter,
a phase detector which refers to the signal of twice the frequency of the alternating current and extracts a second harmonic signal,
a lowpass filter which cancels noise produced in an output signal of the phase detector, and
a signal voltage detecting unit which detects a signal voltage outputted from the lowpass filter.

11. The magnetic sensor according to claim 10, wherein the current supply unit is configured to be able to add a direct current offset component to the alternating current.

12. A magnetic sensor device comprising:
the magnetic sensor according to claim 1; and
a receiving unit which receives information outputted from the magnetic sensor,
wherein an electric activity of a biological cell formed on a substrate is measured by using the information received by the receiving unit.

13. A magnetic sensor device comprising:
the magnetic sensor according to claim 3; and
a receiving unit which receives information outputted from the magnetic sensor,
wherein an electric activity of a biological cell formed on a substrate is measured by using the information received by the receiving unit.

14. A diagnostic device comprising:
the magnetic sensor according to claim 1; and
a receiving unit which receives information outputted from the magnetic sensor,
wherein diagnosis is performed by using the information received by the receiving unit.

15. A diagnostic device comprising:
the magnetic sensor according to claim 3; and
a receiving unit which receives information outputted from the magnetic sensor,
wherein diagnosis is performed by using the information received by the receiving unit.
